(19) United States
(12) Patent Application Publication
(10) Pub. No.: US 2009/0105571 A1
(43) Pub. Date: Apr. 23, 2009

(54) Method and System for Providing Data Communication in Data Management Systems

(75) Inventors: Martin J. Fennell, Concord, CA (US); Lei He, Moraga, CA (US)

Correspondence Address: JACKSON & CO., LLP, 6114 LA SALLE AVENUE, #507, OAKLAND, CA, 94611-2802, US

(73) Assignee: Abbott Diabetes Care, Inc., Alameda, CA (US)

(21) Appl. No.: 11/428,261

(22) Filed: Jun. 30, 2006

(51) Int. Cl. A61B 5/145; H04Q 9/00

(52) U.S. Cl. 600/365; 340/870.07

(57) ABSTRACT

Method and apparatus for providing efficient power management in a data transmitter unit of a data monitoring and management system including a current to frequency conversion unit is provided.

### BACKGROUND

The present invention relates to data monitoring and management systems. More specifically, the present invention relates to method and apparatus for providing improved power management in a data transmission device in data monitoring systems such as analyte monitoring systems.

Continuous analyte (e.g., glucose) monitoring systems including continuous and discrete monitoring systems generally include a small, lightweight battery powered and microprocessor controlled system which is configured to detect signals proportional to the corresponding measured analyte levels using an electrometer, and RF signals to transmit the collected data. One aspect of such analyte monitoring systems include a sensor configuration which is, for example, mounted on the skin of a subject whose analyte level is to be monitored. The sensor cell may use a three-electrode (work, reference and counter electrodes) configuration driven by a controlled potential (potentiostat) analog circuit connected through a contact system.

As with many compact electronic devices, power management is important in maintaining and prolonging the life of the electronic devices. For example, given the structural limitations on the size of a data transmitter unit in analyte monitoring systems, conservation or efficient use and management of the power supply such as a battery is critical in the design of the data transmitter unit.

In view of the foregoing, it would be desirable to provide an approach to improve battery life of electronic devices such as data transmitter units used in data management systems such as analyte monitoring systems.

### SUMMARY OF THE INVENTION

In view of the foregoing, in accordance with the various embodiments of the present invention, there are provided method and system for switching on and off the power supply to the processor of the transmitter unit of a data monitoring and management system, and periodically initiating the processor in an active state for data transmission. In this manner, in one embodiment, power consumption by the transmitter unit of the data monitoring and management system may be improved, extending the battery life of the transmitter unit.

### BRIEF DESCRIPTION OF THE DRAWINGS

FIG. 1 illustrates a block diagram of a data monitoring and management system for practicing one embodiment of the present invention;

FIG. 2 is a block diagram of the transmitter of the data monitoring and management system shown in FIG. 1 in accordance with one embodiment of the present invention;

FIG. 3 is a block diagram illustrating a current to frequency conversion unit of the transmitter of FIG. 2 in one embodiment of the present invention;

FIG. 4 is a flowchart illustrating low power operating mode of the transmitter in accordance with one embodiment of the present invention;

FIG. 5 is a flowchart illustrating a low power operating mode of the transmitter in accordance with another embodiment of the present invention; and

FIG. 6 is a flowchart illustrating a low power operating mode of the transmitter in accordance with still another embodiment of the present invention.

### DETAILED DESCRIPTION

As discussed in further detail below, in accordance with the various embodiments of the present invention, there are provided method and system for power management in data transmission unit in a data monitoring and management system using, for example, a current to frequency conversion unit in the analog interface section of the data transmitter unit for efficient management of the power supply such as a battery.

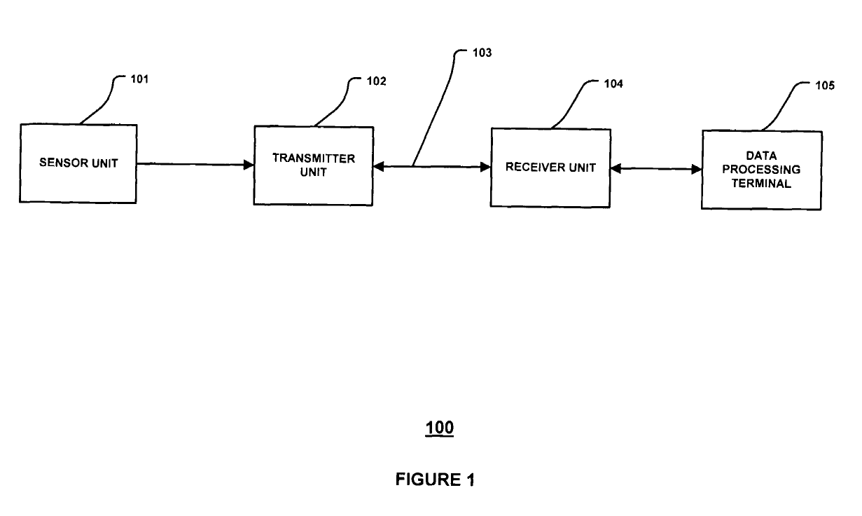

FIG. 1 illustrates a data monitoring and management system such as, for example, an analyte monitoring system 100 for practicing one embodiment of the present invention. In such embodiment, the analyte monitoring system 100 includes an analyte sensor 101, a transmitter unit 102 coupled to the sensor 101, and a receiver unit 104 which is configured to communicate with the transmitter unit 102 via a communication link 103. The receiver unit 104 may be further configured to transmit data to a data processing terminal 105 for evaluating the data received by the receiver unit 104.

Only one sensor 101, transmitter unit 102, communication link 103, receiver unit 104, and data processing terminal 105 are shown in the embodiment of the analyte monitoring system 100 illustrated in FIG. 1. However, it will be appreciated by one of ordinary skill in the art that the analyte monitoring system 100 may include one or more sensor 101, transmitter unit 102, communication link 103, receiver unit 104, and data processing terminal 105, where each receiver unit 104 is uniquely synchronized with a respective transmitter unit 102. Moreover, within the scope of the present invention, the analyte monitoring system 100 may be a continuous monitoring system, or a semi-continuous or discrete monitoring system.

In one embodiment of the present invention, the sensor 101 is physically positioned on the body of a user whose analyte level is being monitored. The sensor 101 may be configured to continuously sample the analyte level of the user and convert the sampled analyte level into a corresponding data signal for transmission by the transmitter unit 102. In one embodiment, the transmitter unit 102 is mounted on the sensor 101 so that both devices are positioned on the user's body. The transmitter unit 102 performs data processing such as filtering and encoding on data signals, each of which corresponds to a sampled analyte level of the user, for transmission to the receiver unit 104 via the communication link 103.

Additional analytes that may be monitored or determined by sensor 101 include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be determined.

In one embodiment, the analyte monitoring system 100 is configured as a one-way RF communication path from the transmitter unit 102 to the receiver unit 104. In such embodiment, the transmitter unit 102 transmits the sampled data signals received from the sensor 101 without acknowledgement from the receiver unit 104 that the transmitted sampled data signals have been received. For example, the transmitter unit 102 may be configured to transmit the encoded sampled data signals at a fixed rate (e.g., at one minute intervals) after the completion of the initial power on procedure. Likewise, the receiver unit 104 may be configured to detect such transmitted encoded sampled data signals at predetermined time intervals. Alternatively, the analyte monitoring system 100 may be configured with a bi-directional RF communication between the transmitter unit 102 and the receiver unit 104, such that both the transmitter unit 102 and the receiver unit 104 are configured to transmit and to receive data over the communication link 103.

Additionally, in one aspect, the receiver unit 104 may include two sections. The first section is an analog interface section that is configured to communicate with the transmitter unit 102 via the communication link 103. In one embodiment, the analog interface section may include an RF receiver and an antenna for receiving and amplifying the data signals from the transmitter unit 102, which are thereafter, demodulated with a local oscillator and filtered through a band-pass filter. The second section of the receiver unit 104 is a data processing section which is configured to process the data signals received from the transmitter unit 102 such as by performing data decoding, error detection and correction, data clock generation, and data bit recovery.

In operation, upon completing the power-on procedure, the receiver unit 104 is configured to detect the presence of the transmitter unit 102 within its range based on, for example, the strength of the detected data signals received from the transmitter unit 102 or a predetermined transmitter identification information. Upon successful synchronization with the corresponding transmitter unit 102, the receiver unit 104 is configured to begin receiving from the transmitter unit 102 data signals corresponding to the user's detected analyte level. More specifically, the receiver unit 104 in one embodiment is configured to perform synchronized time hopping with the corresponding synchronized transmitter unit 102 via the communication link 103 to obtain the user's detected analyte level.

Referring again to FIG. 1, the data processing terminal 105 may include a personal computer, a portable computer such as a laptop or a handheld device (e.g., personal digital assistants (PDAs)), and the like, each of which may be configured for data communication with the receiver via a wired or a wireless connection. Additionally, the data processing terminal 105 may further be connected to a data network (not shown) for storing, retrieving and updating data corresponding to the detected analyte level of the user.

Within the scope of the present invention, the data processing terminal 105 may include an infusion device such as an insulin infusion pump, which may be configured to administer insulin to patients, and which is configured to communicate with the receiver unit 104 for receiving, among others, the measured analyte level. Alternatively, the receiver unit 104 may be configured to integrate an infusion device therein so that the receiver unit 104 is configured to administer insulin therapy to patients, for example, for administering and modifying basal profiles, as well as for determining appropriate boluses (e.g., correction bolus, carbohydrate bolus, dual wave bolus including normal and extended bolus such as square wave bolus, and so on) for administration based on, among others, the detected analyte levels received from the transmitter unit 102.

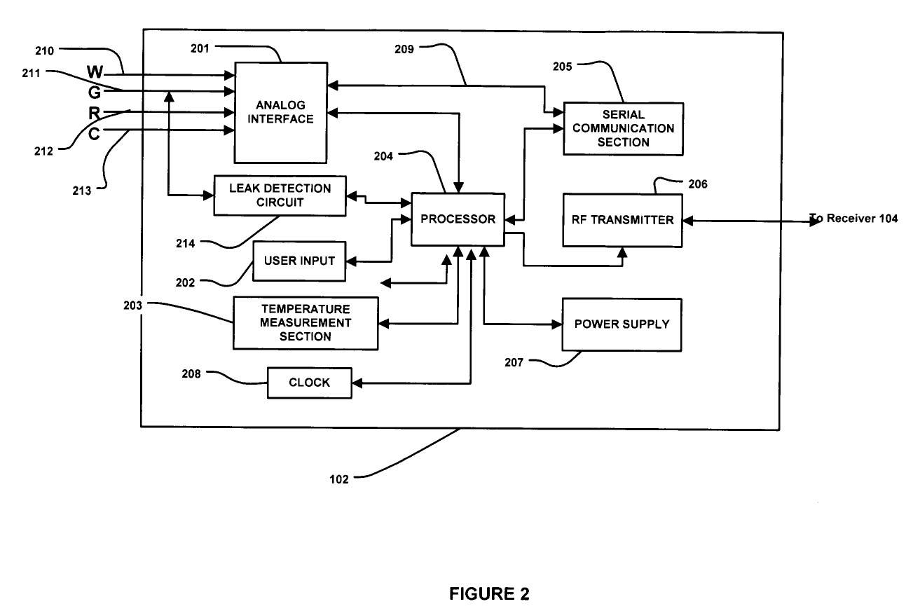

FIG. 2 is a block diagram of the transmitter of the data monitoring and detection system shown in FIG. 1 in accordance with one embodiment of the present invention. Referring to the Figure, the transmitter 102 in one embodiment includes an analog interface 201 configured to communicate with the sensor 101 (FIG. 1), a user input 202, and a temperature detection section 203, each of which is operatively coupled to a transmitter processor 204 such as a central processing unit (CPU). As can be seen from FIG. 2, there are provided four contacts, three of which are electrodes—work electrode (W) 210, guard contact (G) 211, reference electrode (R) 212, and counter electrode (C) 213, each operatively coupled to the analog interface 201 of the transmitter 102 for connection to the sensor unit 201 (FIG. 1). In one embodiment, each of the work electrode (W) 210, guard contact (G) 211, reference electrode (R) 212, and counter electrode (C) 213 may be made using a conductive material that is either printed or etched, for example, such as carbon which may be printed, or metal foil (e.g., gold) which may be etched.

Further shown in FIG. 2 are a transmitter serial communication section 205 and an RF transmitter 206, each of which is also operatively coupled to the transmitter processor 204. Moreover, a power supply 207 such as a battery is also provided in the transmitter 102 to provide the necessary power for the transmitter 102. Additionally, as can be seen from the Figure, clock 208 is provided to, among others, supply real time information to the transmitter processor 204.

In one embodiment, a unidirectional input path is established from the sensor 101 (FIG. 1) and/or manufacturing and testing equipment to the analog interface 201 of the transmitter 102, while a unidirectional output is established from the output of the RF transmitter 206 of the transmitter 102 for transmission to the receiver 104. In this manner, a data path is shown in FIG. 2 between the aforementioned unidirectional input and output via a dedicated link 209 from the analog interface 201 to serial communication section 205, thereafter to the processor 204, and then to the RF transmitter 206. As such, in one embodiment, via the data path described above, the transmitter 102 is configured to transmit to the receiver 104 (FIG. 1), via the communication link 103 (FIG. 1), processed and encoded data signals received from the sensor 101 (FIG. 1). Additionally, the unidirectional communication data path between the analog interface 201 and the RF transmitter 206 discussed above allows for the configuration of the transmitter 102 for operation upon completion of the manufacturing process as well as for direct communication for diagnostic and testing purposes.

As discussed above, the transmitter processor 204 is configured to transmit control signals to the various sections of the transmitter 102 during the operation of the transmitter 102. In one embodiment, the transmitter processor 204 also includes a memory (not shown) for storing data such as the identification information for the transmitter 102, as well as the data signals received from the sensor 101. The stored information may be retrieved and processed for transmission to the receiver 104 under the control of the transmitter processor 204. Furthermore, the power supply 207 may include a commercially available battery.

The transmitter 102 is also configured such that the power supply section 207 is capable of providing power to the transmitter for a minimum of three months of continuous operation after having been stored for 18 months in a low-power (non-operating) mode. In one embodiment, this may be achieved by the transmitter processor 204 operating in low power modes in the non-operating state, for example, drawing no more than approximately 1 μA of current. Indeed, in one embodiment, the final step during the manufacturing process of the transmitter 102 may place the transmitter 102 in the lower power, non-operating state (i.e., post-manufacture sleep mode). In this manner, the shelf life of the transmitter 102 may be significantly improved.

Referring yet again to FIG. 2, the temperature detection section 203 of the transmitter 102 is configured to monitor the temperature of the skin near the sensor insertion site. The temperature reading is used to adjust the analyte readings obtained from the analog interface 201. The RF transmitter 206 of the transmitter 102 may be configured for operation in the frequency band of 315 MHz to 322 MHz, for example, in the United States. Further, in one embodiment, the RF transmitter 206 is configured to modulate the carrier frequency by performing Frequency Shift Keying and Manchester encoding. In one embodiment, the data transmission rate is 19,200 symbols per second, with a minimum transmission range for communication with the receiver 104.

Referring still to FIG. 2, also shown is a leak detection circuit 214 coupled to the guard electrode (G) 211 and the processor 204 in the transmitter 102 of the data monitoring and management system 100. The leak detection circuit 214 in accordance with the various embodiments is configured to detect leakage current in the sensor 101 to determine whether the measured sensor data are corrupt or whether the measured data from the sensor 101 is accurate.

Additional detailed description of the continuous analyte monitoring system, its various components including the functional descriptions of the transmitter are provided in U.S. Pat. No. 6,175,752 issued Jan. 16, 2001 entitled “Analyte Monitoring Device and Methods of Use”, and in application Ser. No. 10/745,878 filed Dec. 26, 2003 entitled “Continuous Glucose Monitoring System and Methods of Use”, each assigned to the Assignee of the present application.

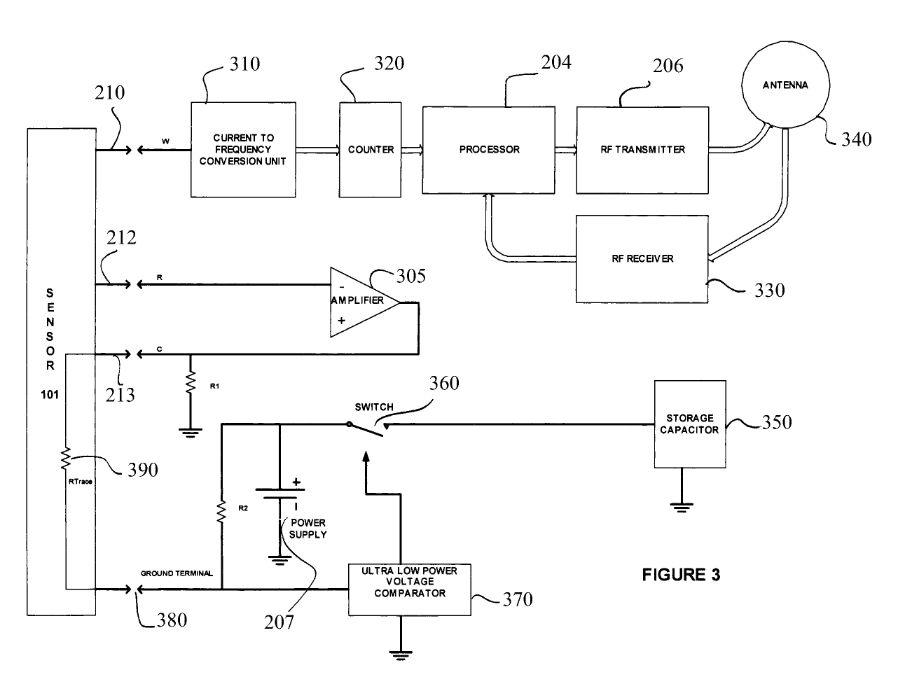

FIG. 3 is a block diagram illustrating a current to frequency conversion unit of the transmitter of FIG. 2 in one embodiment of the present invention. Referring to FIG. 3, there is provided a current to frequency conversion unit 310 operatively coupled to the working electrode 210 (FIG. 2) of the sensor unit 101 (FIG. 1), and is, in one embodiment, configured to convert the received current signal from the working electrode 210 of the sensor unit 101 to a corresponding an output signal whose frequency is associated with the received current signal from the working electrode 210. That is, in one embodiment, the current to frequency conversion unit 310 is configured to generate an output signal that varies in frequency according to the level of the input sensor current signal detected at the working electrode 210 of the sensor unit 101.

Referring to FIG. 3, in one embodiment, the current to frequency conversion unit 310 is configured to operate over a fixed time period, for example, over a 30 second or 60 second period. Also shown in FIG. 3 is a counter 320 coupled to the current to frequency conversion unit 310. In one embodiment, the counter 320 is configured to accumulate a count that is proportional to the frequency of the output signal from the current to frequency conversion unit 310. In other words, in one embodiment, in the fixed time period operating duration for the current to frequency conversion unit 310 signal acquisition period, the counter 320 is configured to generate a count which is proportionally associated with the sensor signal level.

Referring again to FIG. 3, the processor 204 of the transmitter unit 102 in one embodiment is coupled to the counter 320 and is configured to wake up (from an inactive, low power state), once per minute, for example, to retrieve the counter value from the counter 320. The processor 204 is further configured to provide the retrieved or detected counter value to the RF transmitter 206 for transmission to the receiver unit 104 (FIG. 1) via an antenna 340. In one embodiment, the RF transmitter 206 may be configured to operate at a frequency of 433 MHz with frequency shift keying. In this manner, in one aspect, the RF transmitter 206 may be configured to operate for a brief time period during which to transmit the analyte related data received from the processor 204 to the receiver unit 104 over the communication link 103.

Referring back to FIG. 3, there is also provided a storage capacitor 350 which is configured in one embodiment to store energy from the power supply 207 via a low leakage switch 360 (for example, leakage of less than 100 nA) coupled between the power supply 207 and the storage capacitor 350. The storage capacitor 350 in one embodiment may be configured as a low impedance source for electrical current sufficient to power the transmitter 102 during the active data acquisition and transmission phases.

Moreover, as shown in FIG. 3, a voltage comparator 370 (for example, an ultra low power voltage comparator) may be provided and configured to monitor the voltage level on the ground terminal 380. In one embodiment, when the transmitter 102 is coupled to the sensor 101, the RTrace is configured to conduct current and causes the voltage comparator 370 to turn on the switch 360 and supply power to the RF transmitter 206. When the RF transmitter 206 is removed from the sensor 101, the reverse action takes place where the switch 360 is opened, and the power supply 207 is disconnected from the storage capacitor 350. This action removes all power to the transmitter and conserves battery until a new sensor is connected. In one embodiment the battery may last a year or longer.

Referring yet again to FIG. 3, also shown is an amplifier 305 which in one embodiment includes a control amplifier which is configured to control the voltage of the reference electrode 212 of the sensor 101 to provide the appropriate Poise voltage. Moreover, in one embodiment, an RF receiver 330 is provided and which may include a close proximity radio receiver (On/Off Keying (OOK)), operating at, for example at a frequency of 433 MHz, and which may be configured to operate on lower power and to wake up the processor 204 from an inactive state upon detection of any incoming data from the antenna 340.

In one embodiment, using the close proximity radio receiver 330 simple commands may be generated by the receiver 330 and transmitted to the transmitter 206. Examples of commands include, for example, but not limited to commands to initiate a new sensor link, or a temporary transmission on or off commands (for example, during flight in an airplane for FAA compliance.

In this manner, in one embodiment of the present invention, the power supply 207 of the transmitter 102 may be switched on and off by detecting the RF transmitter 206 connection to the sensor unit 101. That is, in one embodiment, the current to frequency conversion unit 310 is configured to operate most of the time, while the processor 204 may be in an inactive (or sleep) mode, and the processor 204 may wake up or (enter active state) just prior to data transmission. Since the current to frequency conversion unit 310 requires very low power to operate, in one aspect, the battery life of the transmitter 102 may be substantially extended.

Accordingly, the current to frequency conversion unit 310 of the analog interface 201 in the transmitter 102 provides for a low power consumption approach, while providing a low pass filter function without substantially engaging the processor 204 functions, and providing a high resolution analog to digital conversion.

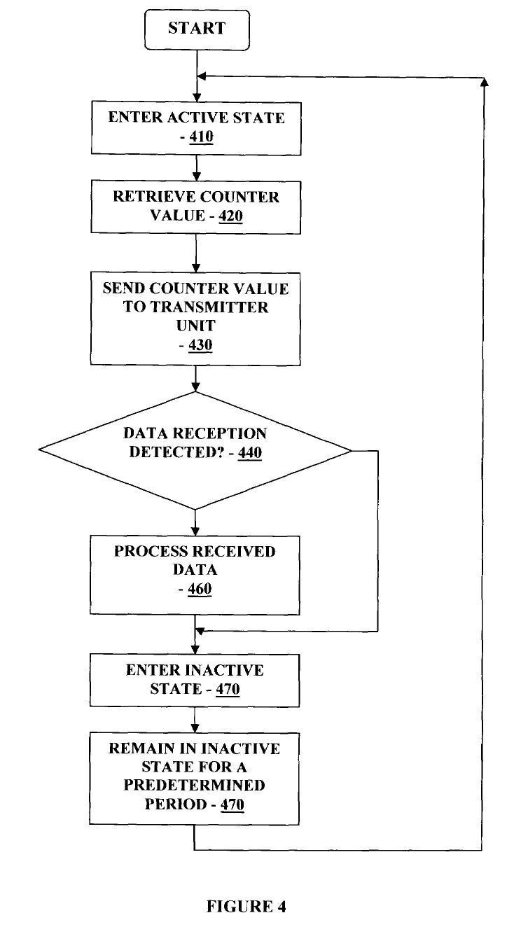

FIG. 4 is a flowchart illustrating low power operating mode of the transmitter in accordance with one embodiment of the present invention. Referring to FIG. 4, the processor 204 (FIG. 3) enters an active state and retrieves a counter value from the counter 320 (FIG. 3) which corresponds to an output signal from the current to frequency conversion unit 310 associated with the detected analyte level from the sensor 101. Thereafter, the retrieved counter value is transmitted to the RF transmitter 206 for data transmission to the receiver unit 104 (FIG. 1) for example, using the antenna 340.

Referring back to FIG. 4, it is determined whether data is received, for example, by the RF receiver 330 of the transmitter unit 102. If data is received, then the processor 204 is configured to process the received data. On the other hand, no data reception is detected, then the processor 204 is configured to enter a low power inactive state. Also, upon processing the received data, the processor 204 is likewise configured in one embodiment to enter the inactive state. Thereafter, the processor 204 in one embodiment is configured to remain in the inactive low power state for a predetermined time period (for example, one minute or less), and thereafter, the routine described above is repeated for the next data transmission.

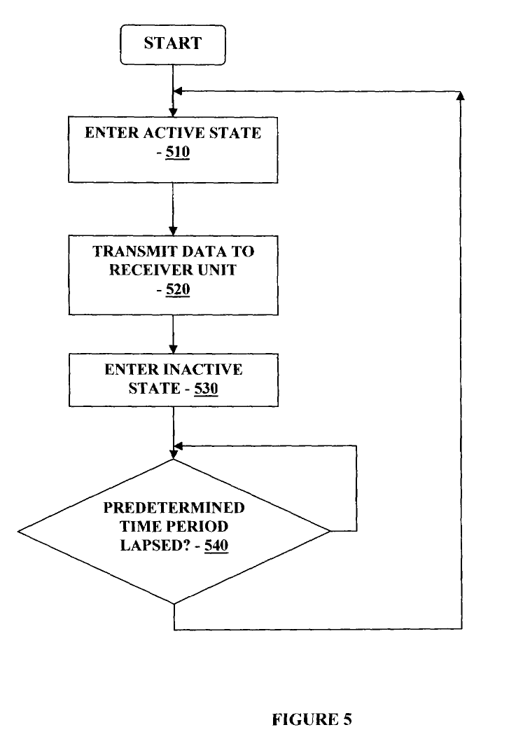

FIG. 5 is a flowchart illustrating a low power operating mode of the transmitter in accordance with another embodiment of the present invention. Referring to FIG. 5, in a further embodiment of the present invention, the processor 204 is configured to enter an active state (for example, at a predetermined time interval, such as once per minute). Thereafter, the processor 204 in active state is configured to transmit data associated with the detected analyte level from the sensor 101 to the receiver unit 104 over the communication link 103. Following the data transmission, the processor 204 is configured to enter an inactive state, until, a predetermined time period has lapsed, at which point, the routine repeats and the processor 204 enters the active state to transmit the next data to the receiver unit 104.

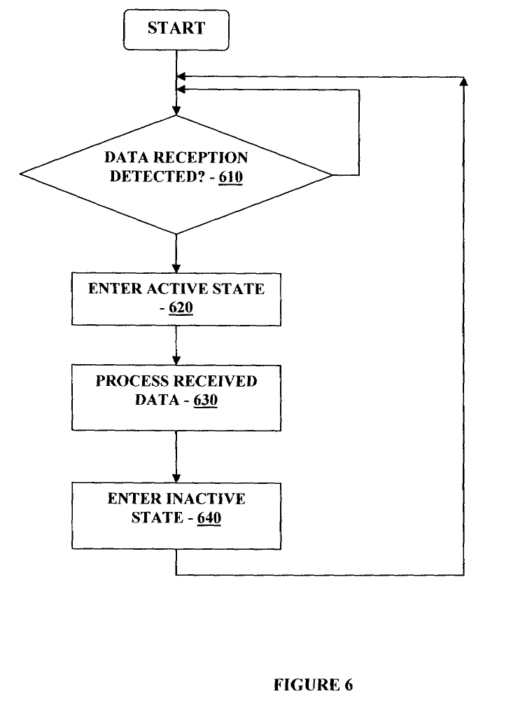

FIG. 6 is a flowchart illustrating a low power operating mode of the transmitter in accordance with still another embodiment of the present invention. Referring to FIG. 6, in one embodiment, the RF receiver 330 in the transmitter unit 102 is configured to detect data reception from the receiver unit 104 at the antenna 340, for example, when data reception is detected, then the processor 204 is configured to enter the active state, drawing power from the power supply 207, for example, and is configured to process the received data. After processing the received data, the processor 204 of the transmitter unit is configured to enter the inactive low power state to conserve battery life.

Accordingly, a data transmitter device in one embodiment includes a data conversion unit, a counter operatively configured to receive an output signal from the data conversion unit, a processor unit operatively coupled to the counter, the processor unit configured to receive a counter output signal, a power supply configured to supply power to the processor unit, and a switch operatively coupled to the power supply and the processor unit, where the switch is configured to establish an electrical connection between the power supply and the processor unit at a predetermined time interval.

The data conversion unit may include a current to frequency conversion unit.

Further, the data conversion unit may be configured to convert an analyte related signal to the output signal having a frequency associated with the level of the analyte related signal.

The device may also include a transmitter operatively coupled to the processor unit, the transmitter configured to transmit a signal associated with the counter output signal, where the transmitter may include an RF transmitter.

In one aspect, the predetermined time interval may be determined by the frequency of data transmission by the transmitter.

A method in accordance with another embodiment includes entering an active operating state, retrieving a counter value, transmitting a signal associated with the retrieved counter value, and entering an inactive operating state, where the transmitted signal is associated with an analyte level of a patient.

The method may also include detecting the analyte level of the patient.

In another aspect, the method may include converting the detected analyte level to an output signal having a frequency associated with the detected analyte level.

Further, the method may also include detecting data reception and processing the received data.

An analyte monitoring system in accordance with yet another embodiment includes a sensor configured for fluid contact with an analyte of a patient, and a transmitter unit, including a current to frequency conversion unit operatively coupled to the sensor, and configured to receive one or more signal associated with the analyte level of the patient, a counter operatively configured to receive an output signal from the current to frequency unit, a processor unit operatively coupled to the counter, the processor unit configured to receive a counter output signal, a power supply configured to supply power to the processor unit, and a switch operatively coupled to the power supply and the processor unit, where the output signal corresponds to the one or more signals associated with the analyte level of the patient, and where the switch is configured to establish an electrical connection between the power supply and the processor unit at a predetermined time interval.

In one aspect, the switch may include a low leakage switch.

In a further aspect, the switch may be configured with a leakage of less than approximately 100 nA.

In another aspect, the system may include a receiver unit operatively coupled to the transmitter unit.

Further, a communication link may be provided operatively coupling the receiver unit and the transmitter unit, where the communication link may include one or more of an RF communication link, a Bluetooth communication link, an infrared communication link, a Zigbee communication link, an 802.1x communication link, and a wired communication link.

In one aspect, the sensor may include an analyte sensor.

The various processes described above including the processes performed by the processor 204 in the software application execution environment in the transmitter unit 102 including the processes and routines described in conjunction with FIGS. 4-6, may be embodied as computer programs developed using an object oriented language that allows the modeling of complex systems with modular objects to create abstractions that are representative of real world, physical objects and their interrelationships. The software required to carry out the inventive process, which may be stored in a memory unit (not shown) of the processor 204, may be developed by a person of ordinary skill in the art and may include one or more computer program products.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

What is claimed is:

1. A data transmitter device, comprising:
a data conversion unit;
a counter operatively configured to receive an output signal from the data conversion unit;
a processor unit operatively coupled to the counter, the processor unit configured to receive a counter output signal;
a power supply configured to supply power to the processor unit, and
a switch operatively coupled to the power supply and the processor unit;
wherein the switch is configured to establish an electrical connection between the power supply and the processor unit at a predetermined time interval.

2. The device of claim 1 wherein the data conversion unit includes a current to frequency conversion unit.

3. The device of claim 1 wherein the data conversion unit is configured to convert an analyte related signal to the output signal having a frequency associated with the level of the analyte related signal.

4. The device of claim 1 further including a transmitter operatively coupled to the processor unit, the transmitter configured to transmit a signal associated with the counter output signal.

5. The device of claim 4 wherein the transmitter includes an RF transmitter.

6. The device of claim 4 wherein the predetermined time interval is determined by the frequency of data transmission by the transmitter.

7. A method, comprising:
entering an active operating state;
retrieving a counter value;
transmitting a signal associated with the retrieved counter value; and
entering an inactive operating state;
wherein the transmitted signal is associated with an analyte level of a patient.

8. The method of claim 7 further including detecting the analyte level of the patient.

9. The method of claim 7 further including converting the detected analyte level to an output signal having a frequency associated with the detected analyte level.

10. The method of claim 7 further including detecting data reception and processing the received data.

11. An analyte monitoring system, comprising:
a sensor configured for fluid contact with an analyte of a patient; and
a transmitter unit, including
a current to frequency conversion unit operatively coupled to the sensor, and configured to receive one or more signal associated with the analyte level of the patient;
a counter operatively configured to receive an output signal from the current to frequency unit;
a processor unit operatively coupled to the counter, the processor unit configured to receive a counter output signal;
a power supply configured to supply power to the processor unit, and
a switch operatively coupled to the power supply and the processor unit;
wherein the output signal corresponds to the one or more signals associated with the analyte level of the patient; and
wherein the switch is configured to establish an electrical connection between the power supply and the processor unit at a predetermined time interval.

12. The system of claim 11 wherein the transmitter includes an RF transmitter.

13. The system of claim 11 wherein the switch includes a low leakage switch.

14. The system of claim 11 wherein the switch is configured with a leakage of less than approximately 100 nA.

15. The system of claim 12 further including a receiver unit operatively coupled to the transmitter unit.

16. The system of claim 15 further including a communication link operatively coupling the receiver unit and the transmitter unit.

17. The system of claim 16 wherein the communication link includes one or more of an RF communication link, a Bluetooth communication link, an infrared communication link, a Zigbee communication link, an 802.1x communication link, and a wired communication link.

18. The system of claim 11 wherein the sensor includes a glucose sensor.
